(19) United States
(12) Patent Application Publication
(10) Pub. No.: US 2024/0165280 A1
(43) Pub. Date: May 23, 2024

(54) METHODS FOR TREATMENT OF CANCERS

(71) Applicant: Fusion Pharmaceuticals Inc., Hamilton (CA)

(72) Inventors: Eric S. Burak, Cambridge (CA); John R. Forbes, Burlington (CA); Ryan W. Simms, Toronto (CA)

(21) Appl. No.: 17/777,850

(22) PCT Filed: Apr. 22, 2022
(86) PCT No.: PCT/US2022/025933

§ 371 Date: Oct. 27, 2022

Related U.S. Application Data
(60) Provisional application No. 63/179,089, filed on Apr. 23, 2021.

(51) Int. Cl. A61K 51/04; A61P 35/00

(52) U.S. Cl. CPC A61K 51/0497; A61P 35/00

(57) ABSTRACT

The present disclosure relates to methods of treating a subject having cancer with an ²²⁵Ac-radiopharmaceutical, which comprises ²²⁵Ac chelated with a compound of Formula I:

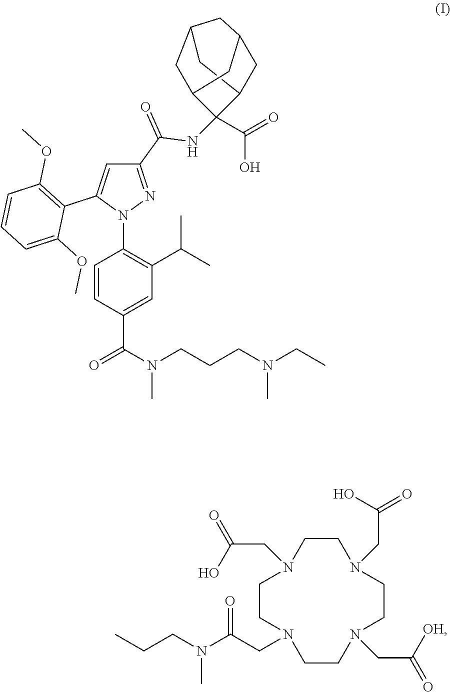

wherein the compound binds to neurotensin receptor 1 (NTSR1) and does not cross the blood-brain barrier, and wherein the ²²⁵Ac-radiopharmaceutical is administered at a dosage of less than 10 MBq/kg of body weight of the subject or is administered as a unitary dosage of less than 40 MBq to the subject.

### RELATED APPLICATION

The present application claims priority to U.S. Provisional Application No. 63/179,089, filed on Apr. 23, 2021, the entire contents of which are hereby incorporated by reference for all purposes.

### BACKGROUND

Neurotensin, a 13 amino acid neuropeptide, is implicated in the regulation of luteinizing hormone and prolactin release and has significant interaction with the dopaminergic system. Neurotensin is bound by neurotensin receptors, which include, among others, neurotensin receptor 1 (NTSR1).

NTSR1 is expressed predominantly in the central nervous system and intestine (smooth muscle, mucosa, and nerve cells). In addition, NTSR1 is highly expressed in a mammalian body and a human body, in particular on several neoplastic cells in several tumor indications, whereas the expression of NTSR1 in most other tissues of the mammalian and the human body is either not existent or low.

Because of this selective expression, NTSR1 is regarded as a suitable target for diagnostic agents, as well as for drugs for treating cancers such as colorectal cancer and pancreatic cancer.

There is a need to develop new methods of using NTSR1 binders for treating cancers.

### SUMMARY

The present disclosure relates to a method of treating cancer, said method comprising administering to a subject in need thereof a therapeutically effective amount of an actinium-225 (²²⁵Ac)-radiopharmaceutical, said ²²⁵Ac-radiopharmaceutical comprising ²²⁵Ac chelated with a compound of Formula I:

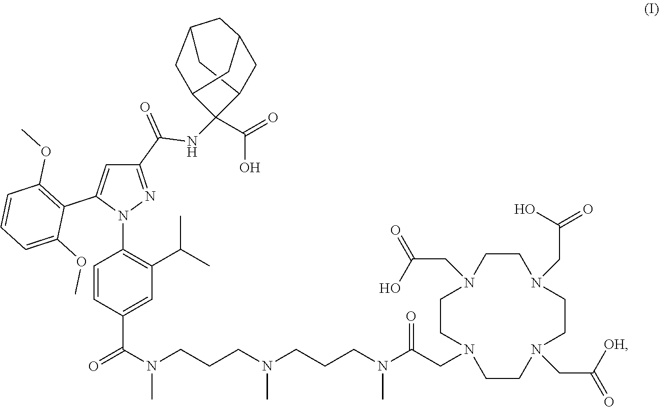 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]

wherein the compound binds to NTSR1 and does not cross the blood-brain barrier, and wherein said ²²⁵Ac-radiopharmaceutical is administered at a dosage of less than 10 MBq/kg of body weight of said subject or is administered as a unitary dosage of less than 40 MBq to said subject.

In some embodiments, said subject is an animal (e.g., mouse) or a human (e.g., patient). In some embodiments, said subject is a human (e.g., patient having cancer).

In some embodiments, said ²²⁵Ac-radiopharmaceutical is administered at a dosage of less than 8 MBq/kg (e.g., less than 6 MBq/kg, less than 4 MBq/kg, less than 2 MBq/kg, less than 1 MBq/kg, or less than 0.5 MBq/kg) of body weight of said subject. In some embodiments, said ²²⁵Ac-radiopharmaceutical is administered at a dosage of less than 1 MBq/kg of body weight of said subject.

In some embodiments, said ²²⁵Ac-radiopharmaceutical is administered at a dosage of less than 250 kBq/kg (e.g., about 225 kBq/kg or less, about 200 kBq/kg or less, about 175 kBq/kg or less, about 150 kBq/kg or less, about 125 kBq/kg or less, about 100 kBq/kg or less, about 75 kBq/kg or less, about 50 kBq/kg or less) of body weight of said subject.

In some embodiments, said ²²⁵Ac-radiopharmaceutical is administered at a dosage of 50-250 kBq/kg (e.g., 50-225 kBq/kg, 50-200 kBq/kg, 50-175 kBq/kg, 50-150 kBq/kg, 50-125 kBq/kg, 50-100 kBq/kg, 75-250 kBq/kg, 75-225 kBq/kg, 75-200 kBq/kg, 75-175 kBq/kg, 75-150 kBq/kg, 75-125 kBq/kg, 75-100 kBq/kg, 100-250 kBq/kg, 100-225 kBq/kg, 100-200 kBq/kg, 100-175 kBq/kg, 100-150 kBq/kg, 100-125 kBq/kg) of body weight of said subject. Each of the dosage can be administered as a single dosing or in multiple times (e.g., 2 times, 3 times, 4 times, or more as needed). In some embodiments, said ²²⁵Ac-radiopharmaceutical is administered at a dosage of 75-225 kBq/kg of body weight of said subject. In some embodiments, said ²²⁵Ac-radiopharmaceutical is administered at a dosage of 100-200 kBq/kg of body weight of said subject.

In some embodiments, said ²²⁵Ac-radiopharmaceutical is administered at a dosage of about 250 kBq/kg, about 225 kBq/kg, about 200 kBq/kg, about 175 kBq/kg, about 150 kBq/kg, about 125 kBq/kg, about 100 kBq/kg, about 75 kBq/kg, or about 50 kBq/kg of body weight of said subject. Each of the dosage can be administered as a single dosing or in multiple times (e.g., 2 times, 3 times, 4 times, or more as needed). In some embodiments, said ²²⁵Ac-radiopharmaceutical is administered at a dosage of about 150 kBq/kg of body weight of said subject.

In some embodiments, said ²²⁵Ac-radiopharmaceutical is administered as a unitary dosage of less than 30 MBq, e.g., 1-28 MBq, 3-25 MBq, 5-20 MBq, 5-15 MBq, or 10-15 MBq to said subject. As used herein, “unitary dosage” typically refers to a single dose. To perform the method of this invention, said ²²⁵Ac-radiopharmaceutical can be administered as a unitary dosage for multiple times (e.g., 2 times, 3 times, 4 times, or more as needed), i.e., administered multiple doses. When said ²²⁵Ac-radiopharmaceutical is administered as a unitary dosage for multiple times, each unitary dosage can be same or different.

In some embodiments, said ²²⁵Ac-radiopharmaceutical is administered as a unitary dosage of less than 20 MBq to said subject. In some embodiments, said ²²⁵Ac-radiopharmaceutical is administered as a unitary dosage of 5-15 MBq to said subject.

In some embodiments, said ²²⁵Ac-radiopharmaceutical is administered as a unitary dosage of about 6 MBq, about 8 MBq, about 10 MBq, about 12 MBq, about 14 MBq, about 16 MBq, or about 18 MBq to said subject.

In some embodiments, said ²²⁵Ac-radiopharmaceutical is administered as a unitary dosage of about 10 MBq to said subject.

In some embodiments, the method of this invention can be used for treating a cancer selected from the group consisting of colorectal cancer, pancreatic ductal adenocarcinoma, small cell lung cancer, prostate cancer, breast cancer, meningioma, Ewing's sarcoma, pleural mesothelioma, head and neck cancer, non-small cell lung cancer, gastrointestinal stromal tumors, uterine leiomyoma, and cutaneous T-cell lymphoma.

In some embodiments, said cancer is selected from one of the following NTSR1-expressing advanced, metastatic and/or recurrent solid tumors: pancreatic ductal adenocarcinoma (PDAC), squamous cell carcinoma of the Head and Neck (SCCHN), colorectal cancer (CRC), gastric cancer, neuroendocrine differentiated (NED) prostate cancer, and Ewing's sarcoma.

In some embodiments, said cancer is colorectal cancer, pancreatic ductal adenocarcinoma, small cell lung cancer, prostate cancer, breast cancer, meningioma, or Ewing's sarcoma.

In some embodiments, said cancer is colorectal cancer or pancreatic ductal adenocarcinoma.

In some embodiments, the method of this invention further includes administration of an imaging agent that comprises a radioactive isotope chelated compound of Formula I as a diagnosis means to determine eligibility of the subject being treated (e.g., a cancer patient) as it relates to NTSR1 expression.

In some embodiments, the radioactive isotope is selected from the group consisting of ⁶²Cu, ⁶⁴Cu, ⁶⁷Cu, ⁶⁷Ga, ⁶⁸Ga, ⁸⁹Zr, ^{99m}Tc, and ¹¹¹In. In certain embodiments, the radioactive isotope is ⁶⁸Ga, ⁸⁹Zr, or ¹¹¹In.

In various embodiments above, said ²²⁵Ac-radiopharmaceutical is administered intravenously.

The details of one or more embodiments of the disclosure are set forth in the description below. Other features, objects, and advantages of the disclosure will be apparent from the below drawing, description, and from the claims.

### BRIEF DESCRIPTION OF THE DRAWINGS

FIG. 1 is a schematic depicting the group average animal weights±standard deviations (g) for [¹⁷⁷Lu]-3BP-227 radiotherapy at 2.1 mCi and 4.5 mCi dosages.

FIG. 2 is a schematic depicting the group average animal weights±standard deviations (g) for [²²⁵Ac]-3BP-227 radiotherapy at 1 μCi and 3 μCi dosages.

FIG. 3 is a schematic depicting the group average animal tumor volumes±standard deviations (mm³) for [¹⁷⁷Lu]-3BP-227 radiotherapy at 2.1 mCi and 4.5 mCi dosages. Tumor volume (TV) was calculated as TV=0.52×(L×W²) where L (length) is the longest diameter of the tumor and W (width) is the shortest diameter of the tumor.

FIG. 4 is a schematic depicting the group average animal tumor volumes±standard deviations (mm³) for [²²⁵Ac]-3BP-227 radiotherapy at 1 μCi and 3 μCi dosages. Tumor volume (TV) was calculated as TV=0.52×(L×W²) where L (length) is the longest diameter of the tumor and W (width) is the shortest diameter of the tumor.

FIG. 5 is a schematic depicting the group average percentage relative weights±standard deviations for [¹⁷⁷Lu]-3BP-227 radiotherapy at 2.1 mCi and 4.5 mCi dosages. Percent relative weight was calculated for a given animal as the percentage of its weight at day n compared to its weight at day 0.

FIG. 6 is a schematic depicting the group average percentage relative weights±standard deviations for [²²⁵Ac]-3BP-227 radiotherapy at 1 μCi and 3 μCi dosages. Percent relative weight was calculated for a given animal as the percentage of its weight at day n compared to its weight at day 0.

FIG. 7 is a schematic depicting the group average relative tumor volumes±standard deviations for [¹⁷⁷Lu]-3BP-227 radiotherapy at 2.1 mCi and 4.5 mCi dosages. Relative tumor volume was calculated for a given mouse as the ratio of tumor volume at day n compared to tumor volume at day 0.

FIG. 8 is a schematic depicting the group average relative tumor volumes±standard deviations for [²²⁵Ac]-3BP-227 radiotherapy at 1 μCi and 3 μCi dosages. Relative tumor volume was calculated for a given mouse as the ratio of tumor volume at day n compared to tumor volume at day 0.

FIG. 9 is a schematic depicting the percent tumor regression for [¹⁷⁷Lu]-3BP-227 radiotherapy at 2.1 mCi and 4.5 mCi dosages. Tumor regression was calculated as the percentage of average relative tumor volume of a given therapy group compared to the average relative tumor volume of the control group on a given day.

FIG. 10 is a schematic depicting the percent tumor regression for [²²⁵Ac]-3BP-227 radiotherapy at 1 μCi and 3 μCi dosages. Tumor regression was calculated as the percentage of average relative tumor volume of a given therapy group compared to the average relative tumor volume of the control group on a given day.

FIG. 11 is a schematic depicting the animal survival curves for the [¹⁷⁷Lu]-3BP-227 radiotherapy at 2.1 mCi and 4.5 mCi dosages and [²²⁵Ac]-3BP-227 radiotherapy at 1 μCi and 3 μCi dosages, where survival is the percentage of surviving mice from the initial cohort (n=6).

### DETAILED DESCRIPTION

### Definitions

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context. For example, a dosage of about 100 kBq/kg means a dosage of 100±10% kBq/kg, i.e., 90-110 kBq/kg.

As used herein, the term “bind” or “binding” of a targeting moiety means an at least temporary interaction or association with or to a target molecule, e.g., binding to NTSR1, as described herein.

The term “cancer” refers to any cancer caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas. A “hematologic cancer” or “liquid cancer,” as used interchangeably herein, is a cancer present in a body fluid, e.g., leukemia, lymphoma, and multiple myeloma.

As used herein, the phrases “co-administer,” “administer in combination,” or “combined administration” means that two or more agents are administered to a subject at the same time or within an interval such that there may be an overlap of an effect of each agent on the subject. Thus, two or more agents that are administered in combination need not be administered together. In some embodiments, the two or more agents are administered within 24 hours (e.g., 12, 6, 5, 4, 3, 2, or 1 hour(s) of one another, or within about 60, 30, 15, 10, 5, or 1 minute(s) of one another. In some embodiments, the two or more agents are administered together, e.g., in the same formulation or, e.g., in different formulations but at the same time.

The term “chelate,” as used herein, refers to an organic compound or portion thereof that can be bonded to a central metal or radiometal atom at two or more points.

As used herein, the term “compound,” is meant to include all stereoisomers, geometric isomers, and tautomers of the structures depicted.

The compounds recited or described herein can be asymmetric (e.g., having one or more stereocenters). All stereoisomers, such as enantiomers and diastereomers, are intended unless otherwise indicated. Compounds discussed in the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

The term an “effective amount” of an agent (e.g., ²²⁵Ac-radiopharmaceutical), as used herein, is that amount sufficient to effect beneficial or desired results, such as clinical results, and, as such, an “effective amount” depends upon the context in which it is being applied. For example, in therapeutic applications, an “effective amount” may be an amount sufficient to cure or at least partially arrest the symptoms of the disorder and its complications, and/or to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. An effective amount may be administered by administering a single dose or multiple (e.g., at least two, at least three, at least four, at least five, or at least six) doses.

The term “pharmaceutical composition,” as used herein, represents a composition containing an ²²⁵Ac-radiopharmaceutical described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others.

Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By “subject” is meant a human (e.g., a patient) or a non-human animal (e.g., a mammal).

As used herein, the term “targeting moiety” refers to any molecule or any part of a molecule that is capable of binding to a given target. The term, “NTSR1 targeting moiety” refers to a targeting moiety that is capable of binding to an NTSR1 molecule.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### Methods of Treatment

Neurotensin receptor 1 (NTSR1) is a validated cancer target known to be upregulated in multiple solid tumor types including colorectal and pancreatic cancers with limited treatment options and unmet medical need. Targeted alpha therapy (TAT) enables delivery of high energy alpha particle emitting isotopes (e.g., Actinium-225 or ²²⁵Ac) to the targeted tumor cells. The present disclosure provides the preclinical therapeutic efficacy studies of a NTSR1-targeted therapeutic agent, i.e., ²²⁵Ac-radiopharmaceutical (hereafter also referred as [²²⁵Ac]-3BP-227) comprising ²²⁵Ac chelated to the compound of Formula I:

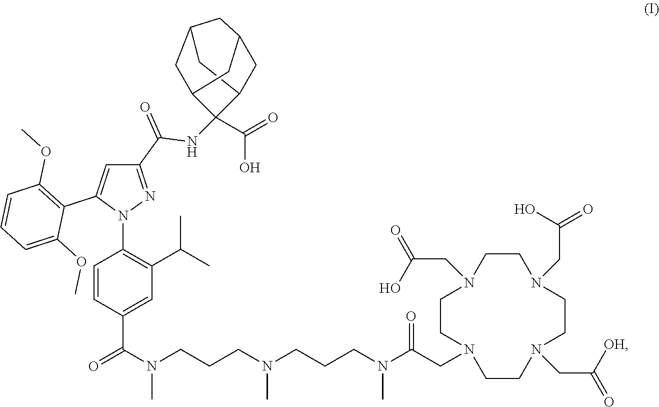 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]

This disclosure provides a direct comparison of [²²⁵Ac]-3BP-227 to [¹⁷⁷Lu]-3BP-227 when tested in preclinical colorectal xenograft model. As used herein, [¹⁷⁷Lu]-3BP-227 refers to ¹⁷⁷Lu-radiopharmaceutical comprising ¹⁷⁷Lu chelated with the compound of Formula I.

Compound of Formula I (also referred as 3BP-227) is a small molecule antagonist targeting NTSR1, which can be radiolabeled with either Actinium-225 or Lutetium-177 to form [²²⁵Ac]-3BP-227 and [¹⁷⁷Lu]-3BP-227, respectively. The synthesis of compound of Formula I, or its metal-chelated radiopharmaceuticals, can be referred to U.S. Pat. No. 10,961,199 B2. For example, [²²⁵Ac]-3BP-227 can be synthesized using the methods described in U.S. Pat. No. 10,961,199 B2.

Compound 3BP-227 can also be radiolabeled with a radioactive isotope to form a radiopharmaceutical suitable for diagnosis or imaging. Examples of the radioactive isotope include, but are not limited to, ⁶²Cu, ⁶⁴Cu, ⁶⁷Cu, ⁶⁷Ga, ⁶⁸Ga, ⁸⁹Zr, ^{99m}Tc, and ¹¹¹In.

In some embodiments, the ²²⁵Ac-radiopharmaceutical comprising ²²⁵Ac chelated to the compound of Formula I is administered parenterally, intravenously, intraarterially, intraperitoneally, subcutaneously, intradermally, trans-gastrointestinally, orally, or locally. In some embodiments, the ²²⁵Ac-radiopharmaceutical comprising ²²⁵Ac chelated to the compound of Formula I is administered via peritumoral injection or intratumoral injection. In certain embodiments, the ²²⁵Ac-radiopharmaceutical comprising ²²⁵Ac chelated to the compound of Formula I is administered intravenously.

The present disclosure also provides method of treating cancer, said method comprising administering to a subject in need thereof a therapeutically effective amount of a pharmaceutical composition comprising ²²⁵Ac-radiopharmaceutical, said ²²⁵Ac-radiopharmaceutical comprising ²²⁵Ac chelated with a compound of Formula I:

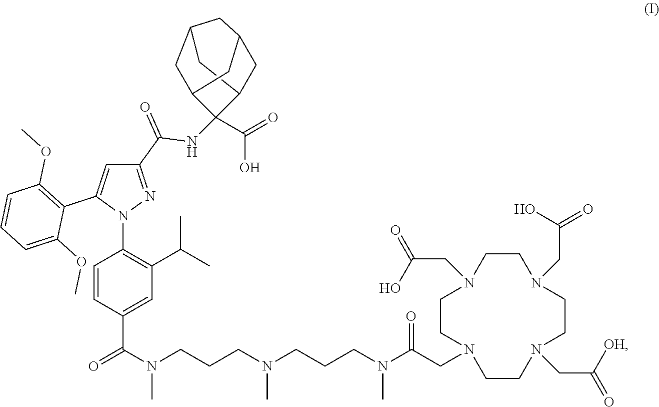 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]

wherein the compound binds to neurotensin receptor 1 (NTSR1) and does not cross the blood-brain barrier, and wherein said ²²⁵Ac-radiopharmaceutical is administered at a dosage of less than 10 MBq/kg of body weight of said subject or is administered as a unitary dosage of less than 40 MBq to said subject.

In some embodiments, the pharmaceutical composition is administered parenterally, intravenously, intraarterially, intraperitoneally, subcutaneously, intradermally, trans-gastrointestinally, orally, or locally. In some embodiments, the pharmaceutical composition is administered via peritumoral injection or intratumoral injection. In certain embodiments, the pharmaceutical composition is administered intravenously.

For therapeutic efficacy studies, single doses of 3,885-8,325 MBq/kg of [¹⁷⁷Lu]-3BP-227 (2,100-4,500 μCO or 1.85-5.55 MBq/kg of [²²⁵Ac]-3BP-227 (1-3 μCO were administered to animals bearing HT29 xenografts (average tumor volume=212±46 mm³), and tumor growth was monitored for 77 days. Mice in the control group were administered vehicle alone (10% ethanol in PBS). Study endpoints included tumor volume measurement and/or impact to animal health status.

Therapeutic efficacy study results indicated a significant and durable tumor growth inhibition in animals treated with 8,325 MBq/kg of [¹⁷⁷Lu]-3BP-227 as compared to the control group. In contrast to that, mice administered 3,885 MBq/kg of [¹⁷⁷Lu]-3BP-227 showed no discernable tumor growth inhibition. Importantly, therapeutic efficacy with [²²⁵Ac]-3BP-227 was unexpectedly far superior to that obtained with [¹⁷⁷Lu]-3BP-227 since durable tumor regression was observed with both low and high treatment doses of [²²⁵Ac]-3BP-227 (1.85 and 5.55 MBq/kg, respectively). There were no treatment-related toxicities observed in any of the studies.

Head-to-head comparison of therapeutic efficacy demonstrated that treatment with [²²⁵Ac]-3BP-227 results in unexpected superior efficacy as compared to [¹⁷⁷Lu]-3BP-227 in a xenograft model of colorectal cancer.

In general, the present disclosure covers use of ²²⁵Ac-radiopharmaceutical in patients with neurotensin receptor 1 (NTSR1)-expressing advanced, metastatic and/or recurrent solid tumors.

The patients typically have histologically and/or cytologically confirmed metastatic or locally advanced unresectable solid tumors that have progressed after treatment with approved therapies or for which no curative therapies are available. For example, patients have one of the following NTSR1-expressing advanced, metastatic and/or recurrent solid tumors: pancreatic ductal adenocarcinoma (PDAC), squamous cell carcinoma of the Head and Neck (SCCHN), colorectal cancer (CRC), gastric cancer, neuroendocrine differentiated (NED) prostate cancer, Ewing's sarcoma.

Single-Photon Emission Computed Tomography (SPECT) imaging following administration of an imaging agent, e.g., ¹¹¹In-chelated compound of Formula I, can be performed as a diagnosis means to determine eligibility as it relates to NTSR1 expression. In some embodiments, eligibility may require sufficient target expression in at least one lesion defined as at least two times the level measured in skeletal muscle. During dose expansion, the imaging criteria may be revised to require a higher tumor to background ratio, or greater number of lesions, if this is deemed appropriate to identify patients more likely to respond to treatment. In some embodiments, a diagnostically effective dose for ¹¹¹In-chelated compound of Formula I (“¹¹¹In-radiopharmaceutical”) for use as a SPECT/CT imaging agent is 185 MBq (or 5 mCi) with less than 4 μCi/kg of mass dose.

When the methods of this invention are used for treating patients having a cancer, patients can receive an injection of ¹¹¹In-radiopharmaceutical during an imaging screening period to assess eligibility per local assessment and to estimate the maximum allowable cumulative dose of ²²⁵Ac-radiopharmaceutical. Significant target expression is defined as at least one measurable lesion with uptake greater than skeletal muscle following ¹¹¹In-radiopharmaceutical administration and SPECT/CT imaging. Planar imaging may be acquired at 4 time points for dosimetry calculation at 0-4, 24±6, 48±6, 72-96 hours after ¹¹¹In-radiopharmaceutical administration. Eligible patients can receive up to 4 cycles of ²²⁵Ac-radiopharmaceutical.

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever.

### Example

### Study Outline

A therapy efficacy study of [¹⁷⁷Lu]-3BP-227 and [²²⁵Ac]-3BP-227 was conducted in mice comprising the following:
1. 30 CD-1 Nude mice (5-6 week-old females; Charles River Laboratories) bearing subcutaneous HT-29 (ATCC #HTB-38; colorectal adenocarcinoma) tumor xenografts distributed amongst 5 groups (n=6). There were two groups each treated with [¹⁷⁷Lu]-3BP-227 or [²²⁵Ac]-3BP-227 and 1 control group (below).
2. Target dose of radioactivity was 2.1 or 4.5 mCi of [¹⁷⁷Lu]-3BP-227 per mouse, or 1 or 3 μCi of [²²⁵Ac]-3BP-227 per mouse, for radiotherapy groups.
3. Mice in the control group were administered vehicle alone (10% ethanol in PBS).
4. For all groups, tumor size and mouse weight were monitored for 34-77 days. Relative tumor volume, relative mouse weight, and tumor regression relative to control were also calculated.

### Experimental Protocol

The experiment was carried out according to protocol “Therapy Study of [¹⁷⁷Lu]-3BP-227 and [²²⁵Ac]-3BP-227 in CD-1 Nude mice bearing HT-29 xenograft tumors” provided below.

### Purpose

Perform an in vivo therapy study to determine the efficacy of [¹⁷⁷Lu]-3BP-227 and [²²⁵Ac]-3BP-227, for the treatment of subcutaneous HT-29 xenograft tumors in nude mice.

### Scope

The compounds to be evaluated are [¹⁷⁷Lu]-3BP-227 and [²²⁵Ac]-3BP-227, which are the ¹⁷⁷Lu and ²²⁵Ac radiolabelled versions, respectively, of the compound of Formula I (also referred as 3BP-227). NTSR1 is overexpressed in several types of cancer, e.g., pancreatic ductal adenocarcinoma (PDAC), Ewing's sarcoma and prostate cancer, but it is rarely expressed in normal tissues. It has demonstrated good uptake and persistence of [¹⁷⁷Lu]-3BP-227 in NTSR1-expressing HT-29 xenograft tumors in Nude mice. It has also demonstrated minimal uptake into 293 tumor xenografts, which do not express NTSR1.

Studies demonstrated that [¹⁷⁷Lu]-3BP-227 was effective as a therapy for suppressing tumor growth of HT-29 xenografts in nude mice for at least 60 days. This effect was particularly evident at a dose of about 4.5 mCi of [¹⁷⁷Lu]-3BP-227 per mouse, while a dose of about 2.1 mCi per mouse somewhat inhibited tumor growth (Study 1). Similar studies were conducted separately (Study 2), but the injection of 4.5 mCi of [¹⁷⁷Lu]-3BP-227 per mouse was not efficacious at suppressing HT-29 tumor growth. However, the average tumor volume at the time of compound injection was significantly larger (about 450 mm³) in Study 2 versus Study 1 (about 200 mm³), which may have masked therapeutic effects. There has no observed acute toxicity in mice with [¹⁷⁷Lu]-3BP-227 therapy.

This study used mice bearing appropriately sized (about 200 mm³) HT-29 tumors. It was also planned to assess the therapeutic potential of [²²⁵Ac]-3BP-227 in the same tumor model. [²²⁵Ac] is an alpha particle labelled version of 3BP-227.

### Study Design

Groups of CD-1 Nude female mice (five groups; n=6 each, 30 total) will be administered radiolabelled test articles via intravenous injection once.

[¹⁷⁷Lu]-3BP-227 will be dissolved in PBS containing 10% ethanol and prepared at a concentration of about 13 mCi/mL and about 25 mCi/mL. [¹⁷⁷Lu]-3BP-227 compounds would be administered (in 200 μL) at a dose of 2.1 mCi (about 13 mCi/mL stock) or 4.5 mCi (about 25 mCi/mL stock) to two groups of animals (n=12 total). [²²⁵Ac]-3BP-227 will be dissolved in PBS containing 10% ethanol and prepared at a concentration of 5 μCi/mL and 15 μCi/mL. [²²⁵Ac]-3BP-227 compounds will be administered (in 200 μL) at a dose of 1 μCi (5 μCi/ml stock) or 3 μCi (15 μCi/mL stock) to two groups of animals (n=12 total). Unlabelled 3BP-227 will be used to mass-match all doses of to 5 nmol. Control mice will be administered 200 μL, of PBS/10% ethanol by tail vein injection (n=6). Mice will be weighed three times a week up to day 28 to monitor therapeutic and toxic effects. The length (L) and width (W) of the tumor will be measured three times a week up to day 28, and the tumor volume will be calculated as per Section 13, Data Calculations and Statistical Analysis.

### Materials

PBS (Life Technologies, 10010-015)
DPBS (Life Technologies, 14287-080)
McCoy's Sa medium (Life Technologies16600-082)+Pen/Strep+10% FBS
Penicillin/Streptomycin (Life Technologies, 15240-062)
FBS (Life Technologies, 12483020)
Alcohol wipes (VWR, CA70000125)
30 g need les {VWR, CABD305106)
26 g need les (VWR, CABD305111)
1 mL slip tip syringes (VWR, CABD309659)
1 mL Luer-lock syringes (VWR, CABD309602)
Mouse restraint
Tungsten syringe shields
Formalin (3.7% formaldehyde buffered in PBS
Digital Calipers (VWR)
Geiger counter (LM 14C)
Dose calibrator (Capintec Inc, CRC-25R)

### Animals and Housing

Thirty-three (33) female CD-1 Nude mice, 3-4 weeks old at delivery and 5-6 weeks old at the study initiation will be ordered from Charles River Laboratories. Animals will be housed 3 per cage in sterile microisolator cages with free access to autoclaved Harlan 8460 chow and water (Clean Level husbandry). Cage enrichment will consist of a PVC tube. Animals will be acclimatized for 5-10 days prior to tumor cell line inoculation.

### Experimental Groups

Group 1: control; n=6
Group 2: 2.1 mCi [¹⁷⁷Lu]-3BP-227; n=6
Group 3: 4.5 mCi [¹⁷⁷Lu]-3BP-227; n=6
Group 4: 1 μCi [²²⁵Ac]-3BP-227; n=6
Group 5: 3 μCi [²²⁵Ac]-3BP-227; n=6

### Tumor Cell Line Inoculations

### Tissue Culture

1. The inoculum for each mouse will consist of 1 million HT-29 cells in a volume of 100 μl. This will be the combination of 75 μL of cells suspended in DPBS and 25 μl of matrigel.

2. Prepare at least twice the number of HT-29 cells in T225 flasks that are required for the day of inoculation (minimum 70 million cells). Cells are grown in McCoy's Sa medium with 10% FBS and penicillin/streptomycin.

3. One day prior to injection, remove Matrigel from the freezer and thaw overnight on ice in the cold room.

4. On the day of injection, place needles and syringes in the freezer. Transfer to ice in the BSC after cell collection.

5. Remove media from the cells and add 10 mL sterile PBS to the flasks; rock gently to rinse cells, aspirate PBS.

6. Add 5 mL of 0.25% Trypsin/EDT A to each flask, ensuring that the growth surface is completely covered. Incubate the cells at 37° C. until detachment from the growth surface (approximately 5 minutes).

7. Add 10 mL of medium to each flask and rinse to remove the cells from the surface. Collect and pool the resuspended cells into a 50 ml conical tube, centrifuge at 400 g for 5 minutes.

8. Aspirate the medium from the cell pellet and resuspend the cells in 1.5 mL of cold DPBS. Maintain on ice. 9. Prepare a dilution of the cells for counting including Trypan blue for live/dead staining. Count on a hemacytometer and calculate the cell concentration. Aliquot 75 million live cells into a new 50 mL conical and adjust the volume of the resuspended cells for a cell concentration of 33 million cells per ml.

10. Add Matrigel to the conical tube at a volume of half the cell suspension volume for a final concentration of 10 million cells per mL. Mix and maintain on ice.

11. Using a pre-chilled 1 mL syringe, gently draw-up the cell suspension to 150 μL line. Cap the syringe with a pre-chilled 30 g needle and remove air from syringe by pushing and pulling plunger. Adjust the plunger to expel excess cell suspension down to a final volume of 100 μL. Place syringe in a plastic bag on ice. Continue this procedure to fill all needles.

### Inoculations

1. Prior to the day of inoculation, earpunch the mice.

2. Working in the animal BSC, prepare a syringe for injection by loosening the cap. Maintain on ice.

3. Remove a mouse from its cage and restrain by hand or with conical restraint.

4. Carefully insert the needle of the syringe just under the skin of the hind flank, tenting to ensure that the needle stays in the subcutaneous space.

5. Inject and record any cases of substantial fluid leakage. Weigh each mouse and record.

### Therapy Procedure

### Prior to Dose Administration

1. Tumor induction will take place over a 7-day period prior to the start of study. Tumor length (L) and width (W) will be measured, using digital calipers, and tumor volume will be calculated (below).

2. Animals will also be weighed as a biomarker of general health.

3. Animals that appear to be in poor health as a result of tumor induction, or show signs of ulcerated or overgrown tumors that effect mobility, will be sacrificed on ethical grounds.

### Therapy Protocol

1. In the days prior to therapy: Prepare and label Luer-lock syringes for injection and prepare the injection log sheet.

2. On the day of radiotherapy administration: weigh the mice and measure the tumors. Calculate tumor volume. Select 30 mice for the therapy study of the 33 inoculated, based on achieving the most uniform group tumor sizes. Assign the mice to experimental groups such that the average tumor volumes of the groups are approximately equal. 3. Set up shielded injection stations.

4. Prepare radiolabelled compounds at working concentrations as described in “Study Design” above. Measure syringes in dose calibrator and record the radioactivity on the log sheet. Place [¹⁷⁷Lu]-3BP-227 syringes in syringe shields for injection.

5. Clean the injection site with alcohol wipes, and inject each animal with 200 μL [¹⁷⁷Lu]-3BP-227, [225 Ac]-3BP-227 or 10% ethanol/PBS buffer in the lateral tail vein; return animal to its cage.

6. Measure and record radioactivity of each syringe after injection. Repeat step 4 until the mice in groups 2-5 have been injected with activity or buffer control.

7. 24 hours post-injection, change all therapy group cages to reduce the potential for efficacy due to excreted radionuclides.

8. Measure and record tumor length (L) and width (W) using calipers three times a week and monitor mouse weight as a biomarker of health.

9. At the end of the prescribed uptake period (day 28), euthanize each animal by cervical dislocation. Collect the tumors, weigh, and place in formal in for fixation.

10. Place animal carcasses in plastic bags and label as radioactive biohazard waste.

### Data Calculation and Statistical Analysis:

1. Tumor volume is calculated according to the formula: TV=(L×W²)×0.52, where L is the longest diameter (length) of the tumor and W is the shortest diameter (width) of the tumor. Tumor volume at day n is expressed as relative tumor volume (RTV) according to the following formula: RTV=TVₙ/TV₀, where TVₙ is the tumor volume at day n, and TV₀ is the tumor volume at day 0. Each mouse weight at day n is expressed as relative mouse weight (RW) according to the following formula: RW=Wₙ/W₀, where Wₙ is the mouse weight at day n, and W₀ is the mouse weight at day 0. Tumor regression (T/C (%)) at day n is determined by calculating RTV according to the following formula: T/C (%)=100×(mean RTV of treated group)/(mean RTV of control group).

2. A spreadsheet is constructed in Excel to calculate tumor volume, relative tumor volume, weight and relative tumor weight, at each time point. Data is graphed in Graphpad Prism.

### Results Summary

A radiotherapy study was conducted with [¹⁷⁷Lu]-3BP-227 or [²²⁵Ac]-3BP-227 in CD-1 Nude mice bearing HT-29 tumor xenografts. The synthesis of two batches of [¹⁷⁷Lu]-3BP-227 was required to conduct the studies described below. The radiochemical yields of both [¹⁷⁷Lu]-3BP-227 batches were 100% and the radiochemical purities of both were 99%. One batch of [²²⁵Ac]-3BP-227 was produced, and the radiochemical yield was 98% and the radiochemical purity was 99%. The analytical summary sheets for testing articles (or radiotracers) are presented in Tables 1-3 below.

TABLE 1
[¹⁷⁷Lu]-3BP-227 analytical summary
sheet for batch 6 (Group 3 injections).

| Radiotracer name | [¹⁷⁷Lu]-3BP-227-06 |
|---|---|
| Batch # | 6 |
| Formula weight (g/mol) | 1303.37 |
| Total activity (mCi) | 49.2 |
| Calibration time | 10:00 |
| Volume of solution (mL) | 1.31 |
| Activity concentration | 37.5 |
| (mCi/mL) | |
| Specific activity (mCi/ | 0.992 |
| nmol) | |
| Formulation | PBS/10% EtOH |
| pH | 7.4 |
| Clarity | clear/colorless |
| Radiochemical yield (%) | 100 |
| Radiochemical purity (%) | 99 |

Analytical HPLC trace shown below

| Peak | Impurity | Impurity | Product | Impurity |
|---|---|---|---|---|
| Peak retention times | 4.4 | 5.2 | 10.84 | 13.96 |
| % Integration | 1.3 | 1.2 | 95.2 | 2.3 |
| Co-injected with cold | no | | | |
| standard | | | | |

TABLE 2
[¹⁷⁷Lu]-3BP-227 analytical summary
sheet for batch 7 (Group 2 injections).

| Radiotracer name | [¹⁷⁷Lu]-3BP-227-07 |
|---|---|
| Batch # | 7 |
| Formula weight (g/mol) | 1303.37 |
| Total activity (mCi) | 36.3 |
| Calibration time | 11:00 |
| Volume of solution (mL) | 0.973 |
| Activity concentration (mCi/mL) | 37.3 |
| Specific activity (mCi/nmol) | 0.994 |
| Formulation | PBS/10% EtOH |
| pH | 7.4 |
| Clarity | clear/colorless |
| Radiochemical yield (%) | 100 |
| Radiochemical purity (%) | 99 |

Analytical HPLC trace shown below

| Peak | Impurity | Product |
|---|---|---|
| Peak retention times | 3.3 | 10.85 |
| % Integration | 0.4 | 99.6 |
| Co-injected with cold standard | no | |

TABLE 3
[²²⁵Ac]-3BP-227 analytical summary sheet
for batch 1 (Groups 4 and 5 Injections).

| Radiotracer name | [²²⁵Ac]-3BP-227-01 |
|---|---|
| Batch # | 1 |
| Formula weight (g/mol) | 1351.37 |
| Total activity (mCi) | 0.097 |
| Calibration time | 10:00 |
| Volume of solution (mL) | 0.97 |
| Activity concentration (mCi/mL) | 0.1 |
| Specific activity | 1 μCi/nmol |
| Formulation | PBS/10% EtOH |
| | (10 mM ascorbate) |
| pH | 7.4 |
| Clarity | clear |
| Radiochemical yield (%) | 98 |
| Radiochemical purity (%) | 99 |

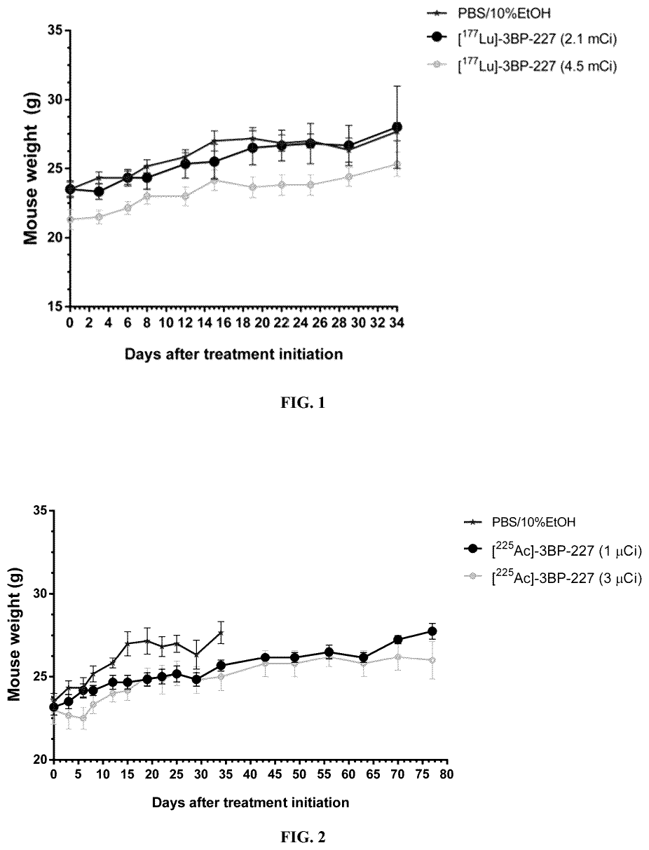

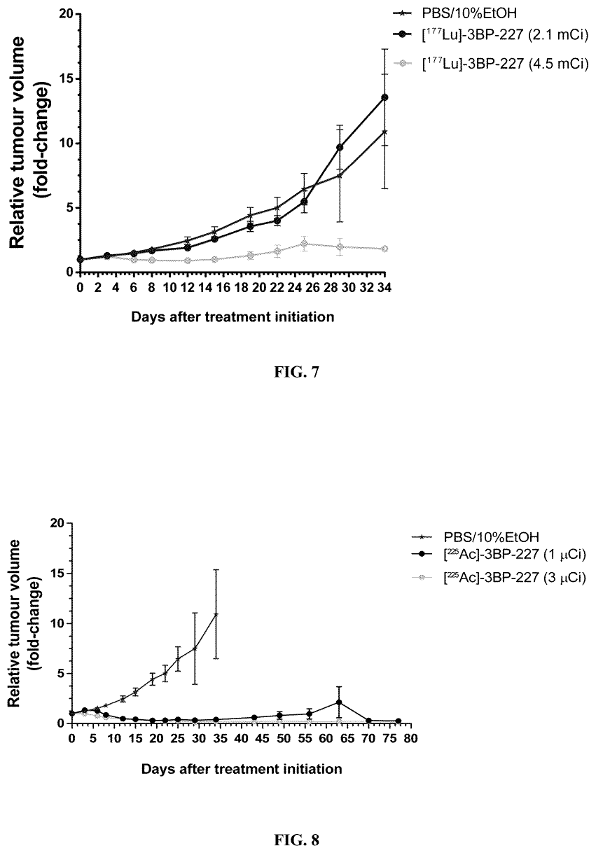

The average weight of the mice at study start was 23±2 g (mean±SD) and the average tumor volume on the day of study start was 212±46 mm³ (mean±SD). Assigned animal numbers, bodyweights, tumor volumes, relative bodyweights, and relative tumor volumes are recorded. Group averages of these data are presented FIGS. 1 to 7. Animals were sacrificed in the [¹⁷⁷Lu]-3BP-227 or PBS/10% ethanol (control) groups on Day 34. Animals receiving [²²⁵Ac]-3BP-227 were sacrificed on Day 77. The dose log for the radiotherapy treatment groups was recorded.

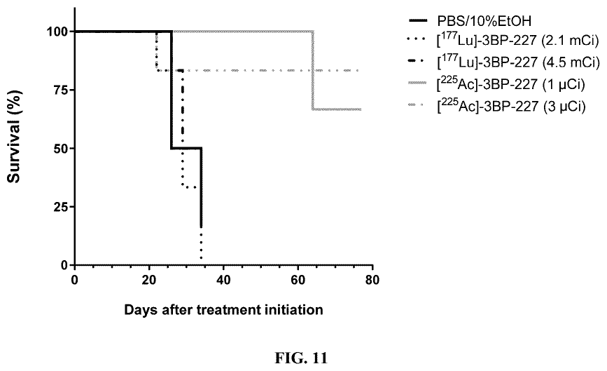

Therapy was generally well-tolerated by mice in the radiotherapy groups. Mice administered [¹⁷⁷Lu]3BP-227 demonstrated an indistinguishable weight gain from those administered vehicle alone (control) (FIG. 6). Mice administered [²²⁵Ac]-3BP-227 therapy also gained weight, albeit to a lesser extent than the controls, over the first 34 days. Sixty-five days after therapy administration, mice in the 3 μCi [²²⁵Ac]-3BP-227 group had a reduced body-weight gain compared to controls (FIG. 6). One mouse in the 3 μCi [²²⁵Ac]-3BP-227 group was euthanized at 22 days post-therapy administration due to poor overall health. Several mice in each of the other 4 groups were also euthanized, but this was due to excessive tumor burden or tumor ulceration, which did not occur in the 3 μCi [²²⁵Ac]3BP-227 group (FIG. 11).

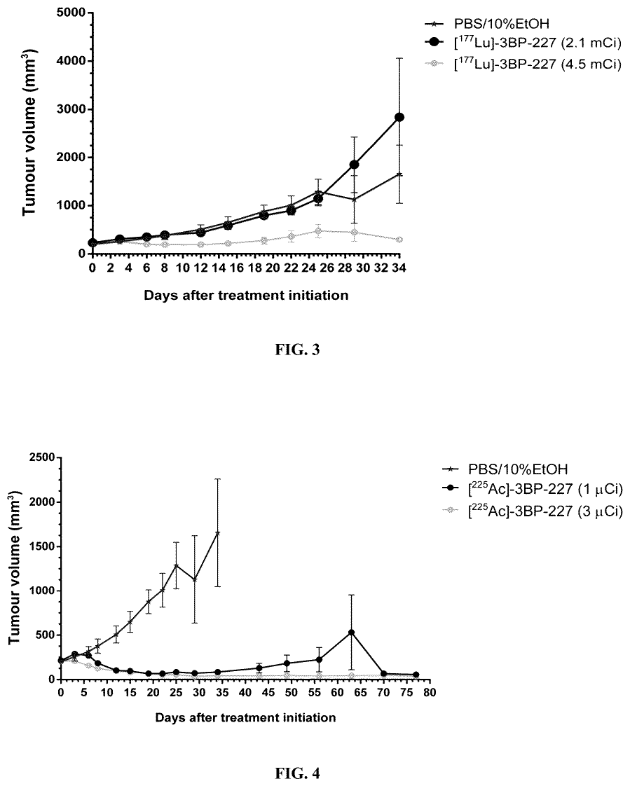

Analysis of the Lu-177 group average relative tumor volume data (FIG. 3) and tumor regression data (FIG. 5) revealed that there was an inhibition in the growth of tumors in the 4.5 mCi [¹⁷⁷Lu]-3BP-227 treatment group relative to controls for the 34 days that the mice were monitored. In contrast, tumors in the 2.1 mCi [¹⁷⁷Lu]-3BP-227 treatment group showed only a brief inhibition in growth (from days 6 to 12).

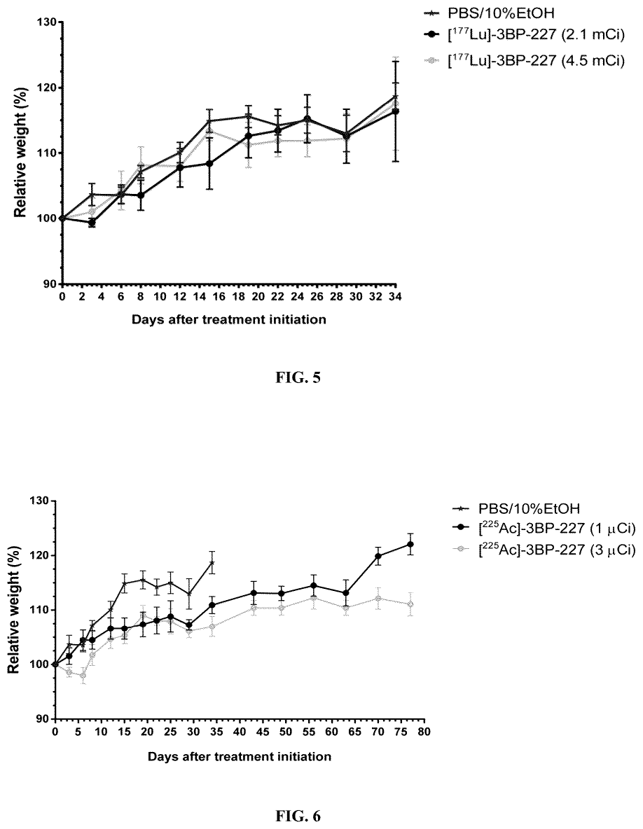

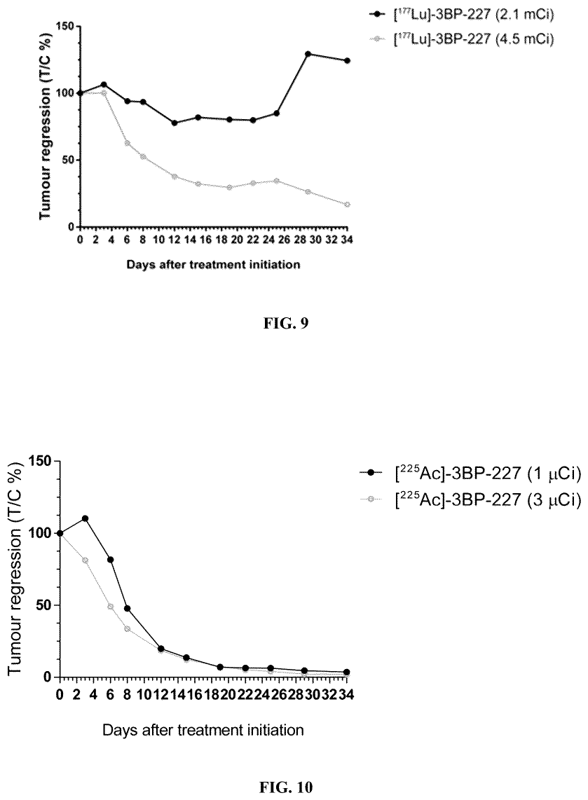

Analysis of the Ac-225 group average relative tumor volume data (FIG. 4) and tumor regression data (FIG. 5) revealed that the tumors regressed in both [²²⁵Ac]-3BP-227 treatment groups. This effect lasted for at least 34 days in the 1 μCi group, after which time, two of six tumors regrew, while in the 3 μCi group, tumor regrowth was not observed. The 3 μCi group also demonstrated a more rapid shrinking of the tumors, which occurred within 6 days of radiotherapy administration (FIG. 10).

Administration of 3 μCi [²²⁵Ac]-3BP-227 was the most efficacious therapy in this study but it was associated with treatment-related morbidity. Both 1 μCi of [²²⁵Ac]-3BP-227 and 4.5 mCi of [¹⁷⁷Lu]3BP-227 were also efficacious at suppressing tumor growth. However, significant tumor regrowth was more frequent in the 4.5 mCi [¹⁷⁷Lu]-3BP-227 versus the 1 μCi [²²⁵Ac]-3BP-227 treatment group. The administration of 2.1 mCi [¹⁷⁷Lu]-3BP-227 was transiently efficacious at inhibiting tumor growth in this model. In summary, the therapy that was most effective while being well-tolerated in this study was 1 μCi of [²²⁵Ac]-3BP-227, followed by 4.5 mCi of [¹⁷⁷Lu]-3BP-227.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

What is claimed is:

1. A method of treating cancer, said method comprising administering to a subject in need thereof a therapeutically effective amount of an ²²⁵Ac-radiopharmaceutical, said ²²⁵Ac-radiopharmaceutical comprising ²²⁵Ac chelated with a compound of Formula I: 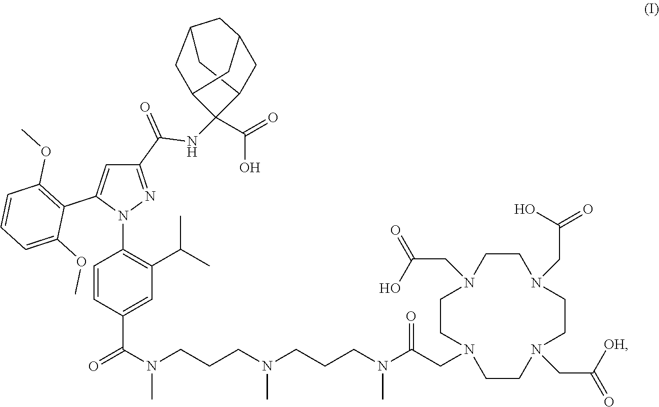 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]
wherein the compound binds to neurotensin receptor 1 (NTSR1) and does not cross the blood-brain barrier, and wherein said ²²⁵Ac-radiopharmaceutical is administered at a dosage of less than 10 MBq/kg of body weight of said subject or is administered as a unitary dosage of less than 40 MBq to said subject.

2. The method of claim 1, wherein said subject is a human.

3. The method of claim 2, wherein said ²²⁵Ac-radiopharmaceutical is administered at a dosage of less than 1 MBq/kg of body weight of said subject.

4. The method of claim 3, wherein said ²²⁵Ac-radiopharmaceutical is administered at a dosage of less than 250 kBq/kg of body weight of said subject.

5. The method of any one of claims 1-4, wherein said ²²⁵Ac-radiopharmaceutical is administered at a dosage of 75-225 kBq/kg of body weight of said subject.

6. The method of any one of claims 1-4, wherein said ²²⁵Ac-radiopharmaceutical is administered at a dosage of 100-200 kBq/kg of body weight of said subject.

7. The method of any one of claims 1-4, wherein said ²²⁵Ac-radiopharmaceutical is administered at a dosage of about 75 kBq/kg, about 100 kBq/kg, about 125 kBq/kg, about 150 kBq/kg, about 175 kBq/kg, about 200 kBq/kg, or about 225 kBq/kg of body weight of said subject.

8. The method of claim 7, wherein said ²²⁵Ac-radiopharmaceutical is administered at a dosage of about 150 kBq/kg of body weight of said subject.

9. The method of claim 2, wherein said ²²⁵Ac-radiopharmaceutical is administered as a unitary dosage of less than 20 MBq to said subject.

10. The method of claim 9, wherein said ²²⁵Ac-radiopharmaceutical is administered as a unitary dosage of 5-15 MBq to said subject.

11. The method of claim 9, wherein said ²²⁵Ac-radiopharmaceutical is administered as a unitary dosage of about 6 MBq, about 8 MBq, about 10 MBq, about 12 MBq, about 14 MBq, about 16 MBq, or about 18 MBq to said subject.

12. The method of claim 9, wherein said ²²⁵Ac-radiopharmaceutical is administered as a unitary dosage of about 10 MBq to said subject.

13. The method of claim 1, wherein said cancer is selected from the group consisting of colorectal cancer, pancreatic ductal adenocarcinoma, small cell lung cancer, prostate cancer, breast cancer, meningioma, Ewing's sarcoma, pleural mesothelioma, head and neck cancer, non-small cell lung cancer, gastrointestinal stromal tumor, uterine leiomyoma, and cutaneous T-cell lymphoma.

14. The method of claim 13, wherein said cancer is colorectal cancer, pancreatic ductal adenocarcinoma, small cell lung cancer, prostate cancer, breast cancer, meningioma, or Ewing's sarcoma.

15. The method of claim 13, wherein said cancer is colorectal cancer or pancreatic ductal adenocarcinoma.

16. The method of claim 1, further comprising administering to the subject an imaging agent that comprises a radioactive isotope chelated compound of Formula I.

17. The method of claim 16, wherein the radioactive isotope is selected from the group consisting of ⁶²Cu, ⁶⁴Cu, ⁶⁷Cu, ⁶⁷Ga, ⁶⁸Ga, ⁸⁹Zr, ^{99m}Tc, and ¹¹¹In.

18. The method of claim 16, wherein the radioactive isotope is ⁶⁸Ga, ⁸⁹Zr, or ¹¹¹In.

19. The method of any one of claims 1-18, wherein said ²²⁵Ac-radiopharmaceutical is administered intravenously.
